(19) United States
(12) Patent Application Publication
(10) Pub. No.: US 2014/0134609 A1
(43) Pub. Date: May 15, 2014

(54) ENZYMATIC METAL NANOPARTICLE SENSOR FOR DETECTING DNA BINDERS

(71) Applicant: Agency for Science, Technology and Research, Singapore (SG)

(72) Inventors: Yen Nee Tan, Singapore (SG); Xiaodi Su, Singapore (SG)

(73) Assignee: Agency for Science, Technology and Research, Singapore (SG)

(21) Appl. No.: 13/968,930

(22) Filed: Aug. 16, 2013

(30) Foreign Application Priority Data
Aug. 17, 2012 (SG) 201206148-7

(51) Int. Cl. C12Q 1/34

(52) U.S. Cl. CPC C12Q 1/34
USPC 435/6.1

(57) ABSTRACT

The present invention provides a colorimetric method for detecting a polynucleotide strand binding molecule using one type of metal particles modified with a single type of interacting molecules. The interacting molecule is capable of specifically binding to nucleic and of protecting the metal particle from aggregation. Furthermore, the metal particles are capable of aggregation upon salt aggregation and/or cleavage of the interacting molecule, and colorimetric change changes upon aggregation.

### CROSS-REFERENCE TO RELATED APPLICATIONS

This application claims the benefit of priority of SG provisional application No. 201206148-7, filed Aug. 17, 2012, the contents of it being hereby incorporated by reference in its entirety for all purposes.

### FIELD OF THE INVENTION

The present invention refers to the field of biochemistry and nanotechnology. More particularly, the invention relates to colorimetric methods of detecting binding of a molecule capable of binding to a nucleic acid that is linked to metal particles.

### BACKGROUND OF THE INVENTION

Determining nucleic acid binding molecules and their sequence specificity is a non-trivial task. Interactions between polynucleotides strand binding molecules and their target sequence are indispensable for many essential cellular processes, such as, for example, gene transcription, replication, recombination and control of gene expression in general. The consequences of these interactions are numerous at the molecular, cellular and physiological level.

For example, cellular mechanisms including control of cell cycle, cell death, signal transduction in response to various stimuli and cell metabolism in general may be affected by such interactions. In turn, such interactions may also be involved in diseases such as for example, immune diseases, cancer and diabetes.

It is therefore desirable to develop simple, specific and robust methods to identify, or detect nucleic acid binding molecules and monitor their binding activity. It is also of interest if such methods are amenable to high-throughput screening techniques.

Conventional methods for detecting and measuring nucleic acid binding molecules include for example, DNAse I footprinting, exonuclease III footprinting, southwestern blotting, various chemical protection and interference assays, ultraviolet cross-linking, gel-shift assay such as electrophoretic mobility-shift assay (EMSA), Chromatin immunoprecipitation (ChIP) assays including ChIP on chip and ChIP-Seq, DNA pull-down assays or microplate capture assays which are a hybrid of the DNA-pulldown assay and Enzyme-Linked Immusorbent assay (ELISA). Such methods are often limited by the availability of targets, complicated, labor-intensive and time-consuming. Moreover, these assays are mainly useful to detect DNA-protein interactions and are not suited for rapid screening of large numbers of samples or multiple nucleic acid binding molecules simultaneously.

More recently, nanomaterials have been used in biosensing. In particular, the unique properties of noble metal nanoparticles have allowed for the development of new biosensing platforms with enhanced capabilities in the specific detection of bioanalytes. For example, noble metal nanoparticles show unique physicochemical properties (such as ease of functionalization via simple chemistry and high surface-to-volume ratios) that allied with their unique spectral and optical properties have prompted the development of biosensing platforms.

Most of the reported metal particle based colorimetric assays for detecting molecules that bind to polynucleotides are designed based on a crosslinking mechanism, in which two sets of metal particles-conjugates are linked/assembled (in the presence of analyte) through inter-particle bond formation. This on-particle biorecognition process is slow and as such it can take up to 12 h to observe a shift in color. In addition, such analyte-induced aggregation mechanism tend to produce false positive results caused by destabilizing effects of unrelated molecules that may be present in the reaction buffer, causing aggregation of the metal particles.

Another approach for detecting molecules that bind to polynucleotides involves the enzymes capable of dissociating metal particles aggregates into dispersed particles. For example, the endonuclease (DNAse I) sensor is used to detect the endonucleolytic activity of the enzyme and inhibition. This technology relies on intensive inter-particle hybridization and necessitates cautious monitoring of the melting (dissociation) behaviour of DNA. In addition, due to the inaccessibility of DNA embedded inside the particle aggregates, this assay has limited application and is not advantageous for proteins that are too bulky or lacking the enzymatic cleavage function.

Assays based on the crosslinking aggregation of metal particles are not suitable to detect single-stranded DNA (ssDNA) binding molecules/protein as ssDNA lack a complementary DNA sequence necessary to form the cross-linked network of particle aggregates.

In contrast to the above mentioned cross-linked approaches, non-crosslinking mechanisms employ unmodified metal particles that do not involve inter-particle linkage and may provide a colorimetric response within minutes. The stability of the metal particles is achieved via the controlled loss and/or gain of stabilization forces on the particle surface. For example, the addition of a salt to a solution containing the metal particles may neutralize the surface charges on the metal particles, initiating the particles to aggregate. However, this type of assay is not suitable for detecting the presence of DNA binding molecules in complex biological samples due to the non-specific binding of proteins/analytes on the unmodified surface of the metal particles.

Thus, there is a need to provide methods to detect polynucleotide strand binding molecules that overcome, or at least ameliorate, one or more of the disadvantages described above.

### SUMMARY OF THE INVENTION

Described herein are methods for colorimetric detection of a polynucleotide strand binding molecule that are based at least in part, on a design of metal particles capable of colorimetric changes. Other known detection methods using metal particles may have long incubation periods, may provide false positive results, may involve multiple tedious steps or stringent assay conditions, or may have low specificity. The methods as described herein have been designed to provide for fast, simple and specific detection methods. These methods allow for combination of the specificity found in cross-linking approaches and the rapidity of non-cross-linking methods into a single assay.

In one aspect of the invention, there is provided a colorimetric method of detecting a polynucleotide strand binding molecule, wherein the method may comprise (a) contacting a sample suspected to comprise a polynucleotide strand binding molecule with a solution comprising one type of metal particles to obtain a sample-particle mixture, wherein the metal particle comprises at its surface a single type of interacting molecules capable of specifically binding to a specific polynucleotide strand binding molecule and capable of protecting the metal particle from aggregation; (b) incubating the mixture for a time sufficient to allow binding of the polynucleotide strand binding molecule to the interacting molecule; (c) contacting the mixture with a cleaving molecule capable of cleaving the interacting molecule; (d) incubating the mixture of (c) for a time sufficient to allow cleavage of the interacting molecule; and (e) measuring color differences wherein a color difference compared to the initial color of the mixture due to metal particle aggregation indicates that the nucleotide strand binding molecule is not present in the sample; wherein a salt is either mixed into the mixture together with the sample or is already present in the solution comprising the metal particle or is added together with the cleaving molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings illustrate an embodiment described herein and serve to explain the principles of the disclosed embodiment. It is to be understood, however, that the drawings are designed for purposes of illustration only, and not as a definition of the limits of the invention.

FIG. 1 is a schematic cartoon illustrating Schematic diagram showing the concept of AuNPs-based colorimetric biosensing assay through enzymatic manipulation in (a) absence and (b) presence of DNA-specific proteins (e.g., ERα). Specifically, sample (a) without proteins will show an immediate change of color solution from red to purple (or redshift in spectrum) upon addition of DNase I due to the loss of DNA protection to AuNPs against salt-induced aggregation. In the presence of protein, the sequence-specific protein binding to the DNA sequences on AuNPs will protect the bound region of the DNA from DNase I cleavage, leading to a more stable AuNPs in red color.

FIG. 2 is a pair of scatter plots demonstrating the ability of DNAse I to digest a double stranded DNA attached to gold nanoparticles and the resulting aggregation of the nanoparticles as shown by the shift in color due the loss of electrosteric protection previously provided by the intact linked DNA. FIG. 2 depicts absorption spectra of double-stranded DNA vitellogenin A2 (dsvit) estrogen-responsive elements (ERE) sequences conjugated to gold nanoparticles (AuNPs) in a buffer solution containing 20 mM MgCl₂ and 50 mM KCl in the absence (a) and presence of DNase (5 units/mL) (b) monitored for 30 minutes.

FIG. 3 is a scatter plot demonstrating the dependence of DNase I on the kinetic of color change induced in DNA-conjugated nanoparticles. Specifically, FIG. 3 shows the aggregation kinetics of dsDNA-conjugates AuNPs in the absence (square) and presence of different amount of DNase (a to d). The aggregation extent of the AuNPs is measured by the change in UV-vis absorption peak for the well-dispersed AuNPs at λ=520 nm to a longer wavelength, and is quantified by the absorbance ratio (r) at λ=700 and λ=520 nm (A700/A520), where a lower r value indicates a smaller degree of aggregation which is inversely correlated to the interparticle distance.

FIG. 4 is a scatter plot demonstrating aggregation kinetics of dsvit-conjugates AuNPs in the presence of 20 unit/mL of Dnase in the buffer solution containing 20 mM MgCl₂ and a)0, b) 10, c) 50, and d) 80 mM KCl. The aggregation extent of AuNPs is measured by the absorbance ratio (r) at wavelengths of l=700 nm and l=520 nm (A700/A520) wavelength of the UV-Vis absorption spectrum of each sample.

FIG. 5 is a pair of scatter plots showing (a) absorption spectra of dsvit-conjugated AuNPs in 20 mM MgCl₂ and 50 mM KCl buffer solution in the absence (blue line) and presence (red line) of 40 nM ERα. (b) Detection limit of ERα obtained at 8 minutes upon addition of 20 unit/mL DNase.

FIG. 6 shows a series of color photographs and corresponding scatter plot demonstrating the aggregation kinetics of dsvit-conjugated gold nanoparticles in the presence of ERα and DNAse I in a buffer solution. (a) Color photographs of dsvit-conjugated AuNPs in 20 mM MgCl₂ and 50 mM KCl buffer solution after adding 20 units/mL DNase to the protein mixture containing different amount of BSA and ERα. (b) Aggregation kinetics of dsvit-conjugated AuNPs samples containing the indicated amount of ERα in the presence of 20 unit/mL DNase I in the same buffer solution.

FIG. 7 is a pair of scatter plots showing (a) aggregation profiles of dsvit-AuNPs upon addition of 20 unit/mL DNase to detect ERα-DNA binding in the nuclear extracts obtained from the positive (+) and negative (−) MCF-7 human breast cancer cells; (b) differentiation of MCF (ER+) and MCF (ER−) with 30 nM ERα “spike-in” the nuclear extracts.

FIG. 8 is a pair of scatter plots showing the (a) aggregation kinetics of ssvit-AuNPs with the addition of 20 unit/L DNase in the 20 mM MgC12 and 50 mM KCl buffer solution and (b) UV-vis absorption spectrum of ssvit-AuNPs in the presence of different SSB concentration obtained at 10 mM upon addition of 20 unit/L DNase under same buffer conditions.

### DETAILED DESCRIPTION OF THE PRESENT INVENTION

Metal particles have played an important role in the development of new biosensors to fulfill the demand for more specific and highly sensitive biomolecular diagnostics. The unique physicochemical properties of such metals at the nanoscale have led to the development of a wide variety of biosensors, such as: (i) nanobiosensors for point of care disease diagnosis, (ii) nanoprobes for in vivo sensing/imaging, cell tracking and monitoring disease pathogenesis or therapy monitoring and (iii) other nanotechnology-based tools that benefit scientific research on basic biology. Metal particles may be used for developing biosensors, due to their simplicity, physiochemical malleability and high surface areas.

A range of highly sensitive biosensing methods for nucleic acids, proteins, antibodies, enzymes and other biological molecules have been developed by exploring different physicochemical properties of the noble metal particles, such as localized surface plasmon resonance (LSPR), fluorescence enhancement/quenching, surface-enhanced Raman scattering (SERS), electrochemical activity, etc.

The physical and chemical properties of metal particles can be in principle accurately tuned by controlling anyone of these parameters, but the flexibility and scope of change are highly sensitive to some specific parameters. Among the biosensing methods, the colorimetric approaches are the simplest and most portable and thus one of the most amenable for use in point-of-care diagnostic methods. Advantageously, colorimetric sensors have high sensitivity, are low cost, can be easily read out with the naked eye or concisely be performed with UV/Vis spectrometry instead of the complex instruments.

Accordingly, in the present invention, the inventors designed a colorimetric method to detect a polynucleotide strand molecule using metal particles. Metal particles are important colorimetric reporters because their extremely high visible-region extinction coefficients (10⁸-10¹⁰ M⁻¹ cm⁻¹) are often several orders of magnitude higher than those of organic dyes. In principle, this colorimetric sensing strategy relies on the fact that the dispersed gold nanoparticles solution is red whereas the aggregated gold nanoparticles solution is purple even blue. And, in order to improve the sensitivity and selectivity of colorimetric sensors, engineering gold nanoparticles with functional molecules having high recognition ability is also necessary.

In the following, unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

Units, prefixes, and symbols are denoted in their Systeme International d'Unités (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Accordingly, the terms defined immediately below are more fully defined by reference to the specification in its entirety.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present invention may be used to detect any polynucleotide strand binding molecule in a sample suspected to comprise a polynucleotide strand binding molecule. As used herein, the term “sample” refers to a biological sample from a patient that may include a single cell or multiple cells or fragments of cells or an aliquot of body fluid, taken from the subject, by means including venipuncture, excretion, ejaculation, massage, biopsy, needle aspirate, lavage sample, scraping (buccal), surgical incision or intervention or other means known in the art.

The term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then and then an —OH, then an —H, and so on at the 2′ position of a sugar moiety), and modifications of these kinds of polynucleotides, wherein the attachment of various entities or moieties to the nucleotide units at any position are included.

Thus as used herein, a “polynucleotide strand binding molecule” may be used interchangeably with “nucleic acid binding molecule” and may be any molecule capable of binding a nucleic acid or/and polynucleotide as defined herein comprising but not limited to a protein such as for example, an enzyme, a transcription factor, a receptor, and an antibody; a nucleic acid as defined herein such as a nucleotide, an oligonucleotide, a polynucleotide such as for example, a DNA single or double stranded, an RNA, an aptamer, an siRNA or a peptide nucleic acid; a chemical compound, agent or molecule; an ion such as a cation or an anion; a carbohydrate or a lipid.

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety, or mass label attached to the nucleotide.

As used herein, the term “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA” is a DNA that has undergone a molecular biological manipulation.

As used herein, the term “oligonucleotide” refers to a short, single-stranded nucleic acid molecule. in the context of the present invention, to an oligomer or nucleic acid polymer (e.g. ribonucleic acid (RNA) or deoxyribonucleic acid (DNA)) or nucleic acid analogue of those known in the art, for example Locked Nucleic Acid (LNA), or a mixture thereof. This term includes oligonucleotides composed of naturally occurring nucleobases, sugars and internucleoside (backbone) linkages as well as oligonucleotides having non-naturally-occurring portions which function similarly or with specific improved functions. A fully or partly modified or substituted oligonucleotide is often preferred over native forms because of several desirable properties of such oligonucleotides such as for instance, the ability to penetrate a cell membrane, good resistance to extra- and intracellular nucleases, high affinity and specificity for the nucleic acid target. Methods of modifying oligonucleotides in this manner are known in the art.

An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

In some oligonucleotides sometimes called oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Examples include, but are not limited to oligonucleotides with phosphorothioate backbones and oligonucleosides with heteroatom backbones, and in particular-CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—]. Also usable are oligonucleotides having morpholino backbone structures.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particular examples include, but are not limited to O[(CH₂)ₙO]mCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]2, where n and m are from 1 to about 10. Other exemplary oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One examplary modification includes 2′-methoxyethoxy (2′-O—CH₂CH₂OCH₃, also known as 2′-O-(2-methoxyethyl) or 2′-MOE) i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-β-dimethylaminoethoxyethyl or 2′-DMAEOE), i.e., 2′-O—CH₂—O—CH₂—N(CH₂)₂, also described in examples hereinbelow.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

Other modifications include 2′-methoxy (2′-O—CH₃), 2′-aminopropoxy (2′-OCH₂CH₂CH₂NH₂), 2′-allyl (2′-CH₂—CH—CH₂), 2′-O-allyl (2′-O—CH₂—CH—CH₂) and 2′-fluoro (2′-F). The 2′-modification may be in the arabino (up) position or ribo (down) position. An exemplary 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases can include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention can include intercalators, reporter molecules, polyamines, polyamides, polyethylene glycols, and polyethers. Typical conjugates groups include cholesterols, lipids, phospholipids, biotin, phenazine, folate, phenan-thridine, anthraquinone, acridine, fluoresceins, rhodamines, coumarins, and dyes.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Oligonucleotides composed of 2′-deoxyribonucleotides (oligodeoxyribonucleotides) are fragments of DNA and are often used in the polymerase chain reaction, a procedure that can greatly amplify almost any small amount of DNA. There, the oligonucleotide is referred to as a primer, allowing DNA polymerase to extend the oligonucleotide and replicate the complementary strand.

The term “polyribonucleotide” refers to a polynucleotide comprising two or more modified or unmodified ribonucleotides and/or their analogs. The term “polyribonucleotide” is used interchangeably with the term “oligoribonucleotide.”

The term “ribonucleotide” and the phrase “ribonucleic acid” (RNA), refer to a modified or unmodified nucleotide or polynucleotide comprising at least one ribonucleotide unit. A ribonucleotide unit comprises a hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

As defined herein, aptamers are nucleic acid ligands which have the property of binding specifically to a desired target compound or molecule or a nucleic acid target through non-Watson-Crick base pairing.

The colorimetric method as described herein may comprise contacting a sample suspected to comprise a nucleic acid binding molecule with a solution that may comprise one type of metal particle to obtain a sample-particle mixture, wherein the metal particle may comprise at its surface a single type of interacting molecules capable of specifically binding to the nucleic acid binding molecule and capable of protecting the metal particle from aggregation. As used herein the term “colorimetric” and grammatical variants thereof refer to the physical description and quantification of the color spectrum including the human color perception spectrum (i.e. visible spectrum).

The solution as defined herein may comprise a colloidal solution comprising one type of metal particle. The solution may be a liquid medium comprising ionic species. As defined herein “colloidal” is a state of subdivision such that the molecules or polymolecular particles dispersed in a medium have at least one dimension between approximately 1 nm and 1 μm, or that in a system discontinuities are found at distances of that order. The colloidal stability of metal particles is governed by the forces that are involved in interaction of colloidal particles. For example, forces may comprise but are not limited to excluded volume repulsion, electrostatic interaction, van der Waals forces, entropic forces, steric or electrosteric repulsion forces. The solution may be a hydrocolloid solution.

Accordingly, the aggregation of metal particles occur when the repulsive force is overcome by the attractive force, through either crosslinking (i.e., interparticle bond formation via the recognition of the biomolecules conjugated on the metal particles surface) or non-crosslinking (i.e., removal of the stabilizing molecules/charge on the metal particles surface) mechanisms.

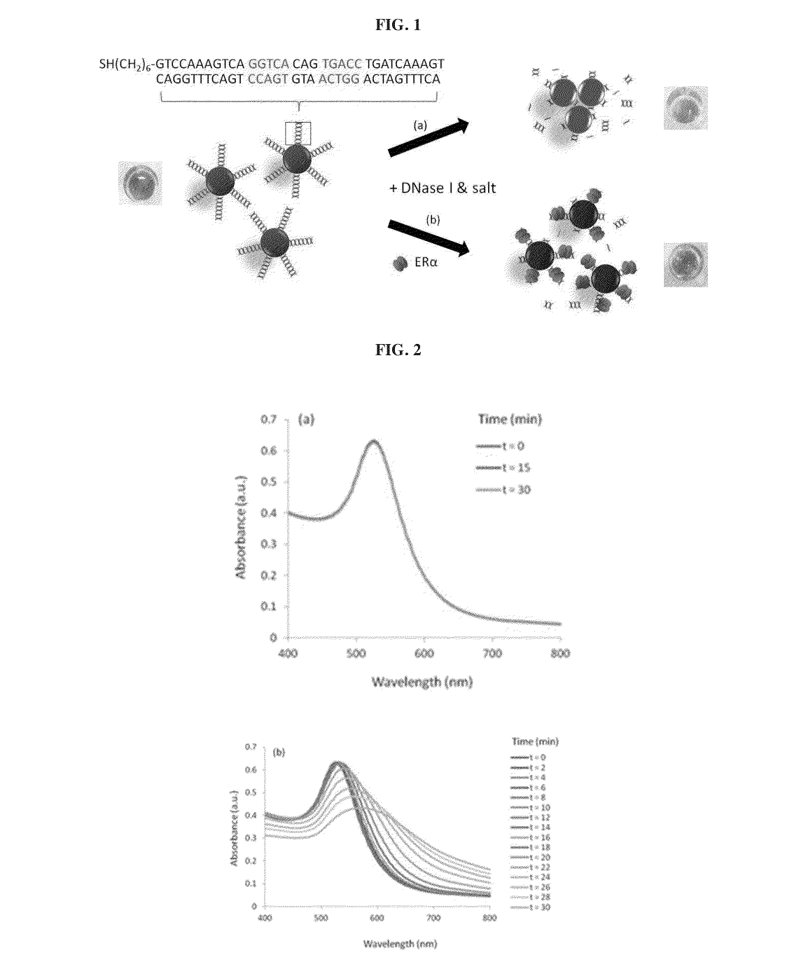

The present inventors surprisingly found that functionalizing metal particles with a single type of interacting molecules prevents the metal particles from aggregating in the presence of salt by maintaining the interparticle distance. That is, the functionalization of metal particle with a single type of interacting molecule provides an electrosteric repulsion force protecting the metal particles from aggregation. By “electrosteric repulsion force” it is a meant a repulsive force provided by the spatial occupation of a molecule that prevents another molecule from occupying the same space (steric hindrance) and by the charge of the molecule since a single type of interacting molecule is provided. In other words, when the metal particles are functionalized with a single type of interacting molecule, the metal particles are protected from aggregation and are dispersed. In some examples, electrosteric protection of the metal particle from aggregation may be provided by functionalizing the metal particle with a molecule comprising but not limited to a nucleic acid, a peptide, a protein, a polymer including polyethylene glycol (PEG) and a charged molecule comprising sodium citrate, a single nucleotide or ATP. In some examples, the interacting molecule may comprise any molecule capable of binding or interacting with a nucleic acid binding molecule as defined herein. For example, FIG. 1 provides an example of gold nanoparticles functionalized with double-stranded DNA.

Additionally the interacting molecules may be capable of binding to metal particles of the invention through covalent interactions such as for example, metal particles-thiolated-interacting molecules conjugates. The interaction between the metal particles and the interacting molecule may be non-covalent. For example, unlabeled single- and double-stranded oligonucleotides have different propensities to adsorb on gold nanoparticles in the colloidal solution. In some examples, the covalent interaction between metal particles and interacting molecules conjugates may be cleavable. In some other examples, the interacting molecule may be internally cleavable. Accordingly, in the absence of cleaving molecules, the interacting molecule is capable of protecting metal particles from salt-induced aggregation by providing electrosteric protection.

In some embodiments, the interacting molecule may be a polynucleotide molecule. In one example as disclosed herein, the polynucleotide molecule may be a double stranded molecule, single stranded molecule, or a triple stranded molecule. The single stranded molecule may be linear or form tertiary structure such as a quadruplex. In some examples, the polynucleotide molecule may comprise but is not limited to a deoxyribonucleic acid (DNA), a ribonucleic acid (RNA), a siRNA, an aptamer, a miRNA or an oligonucleotide comprising natural or modified nucleobases comprising but not limited to a locked nucleic acid, a peptide nucleic acid, a 2′-modified oligonucleotide or a conjugated oligonucleotide as defined above and hereinafter.

In one example, the metal particle is made of a noble metal or alloys of noble metals. In a further example, the noble metal may comprise gold (Au), silver (Ag), palladium (Pd), ruthenium (Ru), rhodium (Rh), osmium (Os), iridium (Ir) or platinum (Pt) or alloys of the aforementioned noble metals. In one example, the noble metal is gold (Au) or silver (Ag). In another example, the metal particle may comprise but is not limited to alloys of noble metals, core/shell bimetallic and trimetallic nanostructures, intermetallic NPs, nanocubes, nanorods, nanowires, nanotubes, nanoplates, alloyed polyhedrons, core/shell polyhedrons, hollow cubes, tadpod-like Au—Pd heterostructure, Au—Pd and Pt—Pd dimer, core/shell nanodendrites and Au@Pt NPs assembling hollow spheres. For example, alloys may comprise bimetallic alloys such as alloys of noble metals such as Au—Ag, Pd₉Au₁ or alloys of noble metals and non-noble metals comprising but not limited to transition metals, such as for example, Co, Ni, Fe, Cu and Mn.

In some embodiments, the metal particle is a nanoparticle or a microparticle. That is the size of the metal particle is about 2 nm to 2000 nm. For example, gold nanoparticles, also known as colloidal gold, may be easily synthesized in sizes ranging between 3 nm and 1000 nm in diameter and in different shapes, being the most common the quasi-spherical shape, mainly due to their surface energy that favors the formation of spherical particles. Generally, the method of choice to synthesize quasi-spherical gold nanoparticles is the chemical reduction of Au(III) to Au(0) ions using sodium citrate as a reducing agent and is known to the skilled artisan.

In this approach, the citrate acts both as reducing agent and as capping agent which, as the metal particles form, prevents the particles from forming larger particles and simultaneously conferring them a mild stability due to electrostatic repulsion between citrate-capped metal particles. It is possible to modify this method to allow a better distribution and control over the size of the metal particles, where a range of about 3 nm, 4 nm, 5 nm, 6 nm, 7 nm, 8 nm, 9 nm, 10 nm, 20 nm, 30 nm, 40 nm, 50 nm, 60 nm, 80 nm, 100 nm, 150 nm, 200 nm, 300 nm, 400 nm, 500 nm and 1000 nm or between about 5 to 1000 nm, about 5 to 500 nm, about 5 to 250 nm, about 5 to 150 nm, about 9 to 120 nm or about 10 to 100 nm can be achieved just by varying the citrate/metal ratio. Alternatively, many aqueous- and organic-based methodologies are known to the person skilled in the art for the controlled synthesis of different noble metal particles, including spherical or non-spherical, pure, alloy or core/shell particles of gold, silver, platinum, palladium and/or rhodium.

The majority of metal particles may exhibit size-related properties that differ significantly from those observed in microparticles or bulk materials. Depending on their size and composition some peculiar properties, such as quantum confinement in semiconductor nanocrystals, surface plasmon resonance in some metal particles and superparamagnetism in magnetic materials.

The method as disclosed herein may comprise incubating the sample-particle mixture for a time sufficient to allow binding of the nucleic acid binding molecule to the interacting molecule. The time may be determined empirically or experimentally to determine the kinetic of binding. Methods known in the art may be used to determine the binding kinetics such as EMSA, gel-filtration, fluorescence (e.g. free resonance energy transfer (FRET), anisotropy and quenching), RNAse, DNase I footprinting, surface plasmon resonance, and affinity resins.

The time sufficient to allow binding may be about 1 second, 2 seconds, 15 seconds, 30 seconds, 45 seconds, 60 seconds, 90 seconds, 120 seconds, 200 seconds, 5 minutes, 10 minutes, 20 minutes, 30 minutes, 45 minutes, 60 minutes, 90 minutes, 120 minutes, 3 hours, 4 hours, 5 hours, 6 hours, 12 hours or 24 hours.

The method as disclosed herein further comprises contacting the mixture as described above with a cleaving molecule capable of cleaving the interacting molecule. For example, the cleaving site on the interacting molecule is hindered upon binding by the nucleic acid binding molecule. Accordingly, the cleaving molecule is not capable of cleaving the interacting molecule because of the hindrance created by the nucleic acid binding molecule. Therefore, the metal particles do not aggregate in the presence of salt because the electrosteric protection is provided by the binding of the nucleic acid binding molecule.

In one example, the cleaving molecule may comprise but is not limited to an enzyme, an aptamer, a deoxyribozyme, a small molecule such as for example propargylic or allenic sulfones, enediyne such as dynemicin A or any molecule capable of cleaving the interacting molecule. In one embodiment the cleaving molecule is an enzyme comprising but not limited to a DNAse, a RNAse, a endopeptidase, or an restriction endonuclease. In a further embodiment, the enzyme is a nuclease which may be an endonuclease or an exonuclease. In a further embodiment, the nuclease may comprise but is not limited to Deoxyribonuclease (DNase), a Ribonuclease (RNase), an endonuclease II such as for example, an endo VI or an exo III, a T7 endonuclease and a S1-nuclease. The enzyme may be capable of catalyzing the hydrolytic cleavage of phosphodiester linkages in the DNA backbone or to catalyze the cleavage of RNA into smaller fragments. In one example, the enzyme is a DNAse I. DNAse I is not able to cleave the DNA at the site at which the minor groove is obstructed by the nucleic acid binding molecule wherein nucleic acid binding molecule is a DNA-binding ligands. Thus, the present inventors exploited the retainable stability of AuNPs due to the retarded DNA cleavage upon binding of a nucleic acid molecule with the interacting molecule.

As disclosed herein, the method comprises measuring color differences wherein a color difference compared to the initial color of the mixture due to metal particle aggregation indicates that the nucleotide strand binding molecule is not present in the sample. As explained above, in the absence of a nucleic acid binding molecule and in the presence of an appropriate salt concentration, the interacting molecule that is bound to the metal particles can be cleaved in the presence of a cleaving molecule thereby suppressing the electrosteric repulsion and allowing aggregation of the metal particles.

Aggregation of metal particles influences the resonant frequencies of the particles, and thus results in a shift in the colorimetric properties of the particles. The aggregated state of the particles can be readily detected using, for example, one or more visualization methods, comprising but not limited to one or more of microscopy techniques, dynamic light scattering techniques, visual observation of color, UV-vis absorption techniques, or surface plasmon resonance techniques.

For example, the aggregation state of the particles may be observed using microscopic examination or dynamic light scattering to determine whether the particles are clumped together or are evenly dispersed in the reaction buffer. More rapid detection may be achieved by observation of the color of the reaction buffer by visual inspection, measurement of UV/Vis absorbance, or measurement of localized surface plasmon resonance. In some embodiments, the surface plasmon resonance technique is a localized surface plasmon resonance technique (LSPR).

As explained above, localized surface plasmon resonance (LSPR) is a characteristic of noble metal particles, including noble metal nanoparticles, that is used for biosensing. The LSPR of metal particles arises from the electromagnetic waves that propagate along the surface of the conductive metal. When excited with an electromagnetic wave, such as light, most noble metal particles produce an intense absorption and scattering due to the collective oscillation of the conduction electrons located at the particles' surface. For example, in gold and silver particles, the LSPR yields exceptionally high absorption coefficients and scattering properties within the UV/visible wavelength range that allows them to have a higher sensitivity in optical detection methods than conventional organic dyes. Moreover, their LSPR properties can be easily modulated according to their size, shape and composition.

Example of LSPR modulation through different particles compositions. The LSPR absorption band of gold/silver alloy particles increases to longer wavelengths with increasing amounts of gold. Typically, colloidal solutions of spherical gold nanoparticles (<40 nm) present a red color with their LSPR band centered at about 520 nm, while spherical silver NPs present a yellow color with their LSPR band centered at about 420 nm. Both metals can also be combined in an alloy or core-shell conformation, presenting a LSPR band that can vary within the wavelength limits of pure metal particles LSPR bands.

These LSPR bands are usually weakly dependent on the size of the particles and the refractive index of the surrounding media, but strongly change with inter-particle distance, for example aggregation of particles leads to a pronounced color change as a consequence of the plasmon coupling between particles and a concomitant red-shift of the LSPR absorption band peak. The present invention therefore provides a method to exploit this inter-particle distance color change. On the one hand, this is achieved by functionalizing the particles with interacting molecules capable of providing an electrosteric repulsive force, thus maintaining the distance between the particles. On the other hand, the use of a molecule capable of cleaving the interacting molecules enables the shortening of the distance between the particles and thus their aggregation followed by a color change.

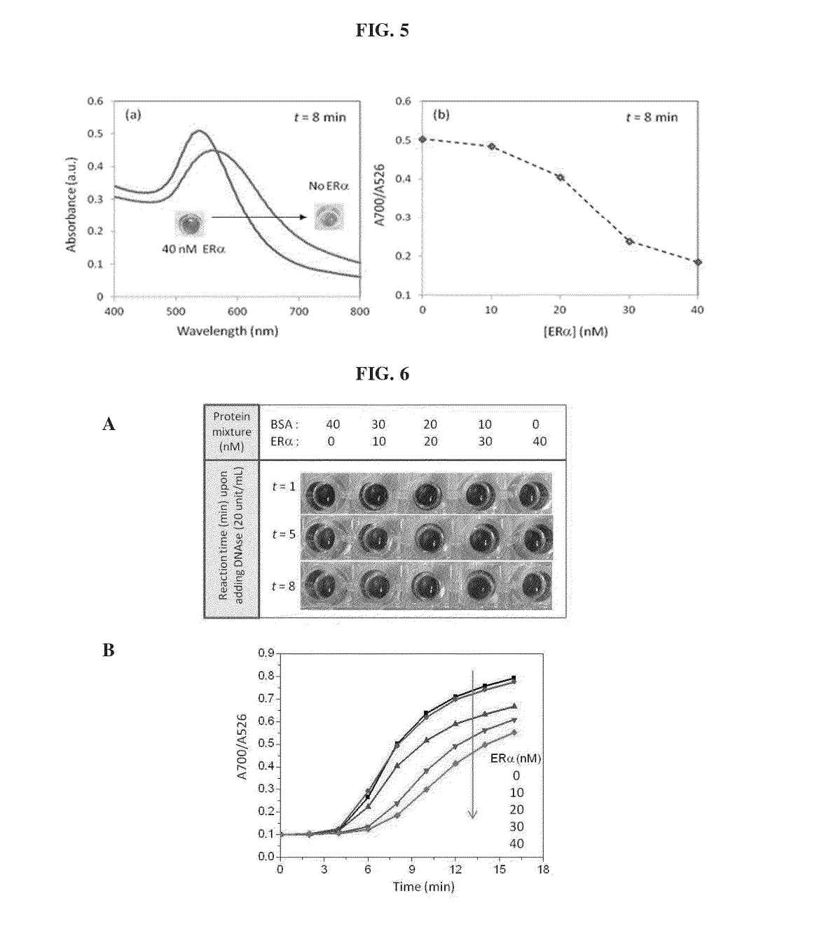

In some embodiments, the detection is a qualitative colorimetric method of detecting a polynucleotide strand binding molecule or a qualitative and quantitative colorimetric method of detecting a polynucleotide strand binding molecule. Some illustrative examples (e.g. example II, FIGS. 5 and 6) below provide qualitative colorimetric methods that allow the specific detection of a polynucleotide strand binding molecule interacting with a specific interacting molecule.

As used herein the term “isolated” biological component (such as a nucleic acid molecule, protein or organelle) has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Quantitative colorimetric method may for example, involve the use of an isolated polynucleotide strand binding molecule of interest of known concentration. The skilled person in the art would know how to, for example, by recombinant expression obtain a purified DNA-binding protein. Once the isolated polynucleotide strand binding protein is obtained, a series of standard of known concentration can be prepared and mixed to the solution containing the metal particles linked to the interacting molecules. A standard curve can then be obtained by measuring the color change according to the concentration of the isolated polynucleotide strand binding protein of interest.

In some embodiments, the aggregation of the metal particles is controlled by the presence of any cleaving molecule that is added to the mixture or reaction buffer that is capable of cleaving the interacting molecule and thus, suppresses the electrosteric protection of the metal particles. In some embodiments, the aggregation of the metal particles is inhibited or at least retarded by the presence of the polynucleotide strand binding molecule. The polynucleotide strand binding molecule is capable upon binding to the interacting molecule to block the cleavage of the interacting molecule by the cleaving molecule.

In some embodiments, a sample that may contain the nucleic acid binding molecule may be added to the mixture or reaction buffer prior to, simultaneously with, or subsequently to the mixing together (or combining or contacting) of the metal particles and the interacting molecule. The nucleic acid binding molecule is capable of binding to (e.g., binds to) the full-length target recognition sequence if added before the cleaving molecule have been added to the solution. As used herein, the term “target recognition sequence” is used in a variety of different forms throughout this document and is defined by the context in which it is used. “Target sequence” and “target site” refer to a sequence (primary, secondary or tertiary) within a molecule to which an interacting molecule shows varying degrees of homology, affinity, or complementarity such that the molecules bind together through this sequence.

When the mixture of the metal particles also contains a nucleic acid binding molecule that is capable of recognizing and binding to the full-length target recognition sequence on the interacting molecule, the nucleic acid binding molecule will bind to full-length target recognition sequences, modulating the cleavage by the cleaving molecule and thus the aggregation state of the metal particles. As indicated above, the nucleic acid binding molecule may act as a steric spacer and a cleaving molecule inhibitor/blocker, resulting in a reduction of aggregation between particles, thus influencing the inter-particle distance and as a result affecting the resonance parameters of the particles.

Thus, in some embodiments, when there is a detectable amount of nucleic acid binding molecule present (e.g., the amount of protein nucleic acid binding molecule is above the lower detection limit of the assay), the presence of the nucleic acid binding molecule will have a detectable effect on the aggregation state of the particles in the presence of a cleaving molecule and salt. The particles will appear less aggregated or even monodispersed in the reaction as compared to the aggregation state created by the cleaving of the interacting molecule in the absence of nucleic acid binding molecule, when observed using for example microscopy techniques or dynamic light scattering techniques. In some embodiments, the metal particle aggregation is obtained via salt aggregation and/or cleavage of the interacting molecule as seen in FIG. 1.

In some embodiments, the methods provided herein include detecting the aggregation state of the metal particles in order to determine whether any nucleic acid binding molecule is present in the sample (and optionally how much), as described above, using visual, colorimetric or other methods to determine the inter-particle distance (e.g., the state of aggregation or dispersion) of the particles in the solution or the reaction buffer. The results of such detection may be compared to the aggregated state of metal particles in the reaction buffer in the absence of the nucleic acid binding molecule of interest.

The binding of the nucleic acid binding molecule to the target recognition is a specific binding event, meaning that the binding is reversible, measurable and saturatable (also referred to as “specific binding” or as one of the nucleic acid binding molecule-binding protein or the nucleic acid target recognition sequence “specifically binding” to the other). Thus, in some embodiments, the binding of the nucleic acid binding molecule to the target recognition sequence may have a specific affinity constant that can be measured. In some embodiments, the concentration of nucleic acid binding molecule in a sample may be determined using titration methods, in order to develop a binding curve. Similarly, in some embodiments, the affinity binding constant between the nucleic acid binding molecule and the target recognition sequence may be measured if the titration is performed using known concentrations of nucleic acid binding molecule and metal particles.

In some embodiments, the specific binding affinity of the nucleic acid binding molecule for various different target recognition sequences may be assessed using the methods provided herein. In some embodiments, the methods may be designed as a competitive assay, for example between a consensus target recognition site on the interacting molecule conjugated to the metal particles and a competitor of the nucleic acid binding molecule may affect the cleavage of the interacting molecule by the cleaving molecule. As will be appreciated, the design of the methods as described herein is general, and the methods may be used to detect, quantify or test the specific binding affinity of any nucleic acid binding molecule that binds to an interacting molecule in a sequence-specific manner. For example, the sequences of the nucleic acid can be readily synthesized to adapt the methods for the nucleic acid binding molecule of interest.

In some embodiments, the interacting molecule may be a double stranded or a single stranded polynucleotide molecule and the polynucleotide strand binding molecule may be a polynucleotide binding protein. In some examples, the interacting molecule may be bound to the surface of the metal particle via a linker, ionic attachment, chemisorption or physical adsorption as explained in more detail below. In some examples, the interacting molecules may be conjugated to the metal particles by a thioether linkage. Such thioether linkages when bound to the metal particle stabilize the metal particles against aggregation. In some embodiments, there is provided the method as described herein, wherein chemisorption is via a functional group which bind the metal particle, wherein the functional group selected from the group consisting of thiols and peptide sequences comprising cysteine and thiolated amino acids. The presence of a thiol group in cysteine may be used to bind a peptide sequence comprising at least one or more exposed cysteine to the metal particle, such as gold particles. The peptide sequence may also comprise modified amino acid comprising a thiol group. In a further embodiment, there is provided the method described herein, wherein physical adsorption comprises binding of peptide sequences comprising simultaneous acidic and basic amino acids groups.

For example, in case the metal particle is a colloidal gold, the gold nanoparticle may be stabilized against aggregation by using long hydrocarbon ligand chains consisting of various functional groups. One end of these molecules is adsorbed on the gold surface, whereas the other end points towards the solution. For example, mercaptocarboxylic acid molecules may be adsorbed on a gold particle surface. Examples of water-soluble metal particles include carboxylic acids as functional groups which stabilize the gold particles by electrostatic repulsion. Such carboxylic groups may further be exploited for the conjugation of other molecules to the particles.

The choice of ligand may depend on the particle size and the solvent. The ligands may also be used as anchor points for further attachment of biological molecules. For example, mercaptocarboxylic acids may be used to stabilize the gold particles due to the strong affinity of sulfur for gold. Thiolated interacting molecules can directly be bound on gold particles surface via thiol-gold affinity interactions. For example, aptamer-gold nanoparticles may be created for recognition of nucleic acid binding molecules. Other examples of interacting molecules that may replace some of the original stabilizer molecules (i.e. thioether linkage) when they are added directly to the particle solution comprise interacting molecules that have a functional group which can bind to the metal surface, such as gold surface (like thiols or specific peptide sequences). Accordingly, in some examples, interacting molecules may comprise but are not limited to oligonucleotides, peptides, and PEG that may be linked to metal particles, such as gold particles.

Accordingly, in some embodiments, the metal particles and the interacting molecules are conjugated through a thioether linkage. In one example, the metal particles may be modified with a thiol linker that is carbon-bonded sulfhydryl group (—C—SH or R—SH) wherein R represents a unsubstituted or substituted group comprising but not limited to an aliphatic or aromatic alkane, an alkene or other carbon-containing group of atoms. In a further example, the thiol containing linker may comprise but is not limited to C₁-C₂₀ alkyl thiol comprising but not limited to 1-propanethiol, 1-butanethiol, 1-pentanethiol, 1-hexanethiol, 1-heptanethiol, 1-octanethiol, 1-nonanethiol, 1-decanethiol, 1-undecanethiol, 1-dodecanethiol, 1-tetradecanethiol, 1-pentadecanethiol, 1-hexadecanethiol, 1-octadecanethiol, 2-ethylhexanethiol, 2-methyl-1-propanethiol, 3-methyl-1-butanethiol, butyl 3-mercaptopropionate, tert-dodecylmercaptan, and tert-nonylmercaptan; dithiols comprising but not limited to 1,2-ethandithiol, 1,3-propanedithiol, 1,4-butanedithiol, 2,3-butanedithiol, 1,5-pentanedithiol, 1,6-hexanedithiol, 1,8-octanedithiol, 1,9-nonanedithiol, tetra(ethylene glycol) dithiol, hexa(ethylene glycol) dithiol, 2,2′-(ethylenedioxy)diethanethiol, and 5,5′-bis(mercaptomethyl)-2,2′-bipyridine; functionalized thiols comprising but not limited to (1′-mercaptoundecyl)-N,N,N-trimethylammonium bromide, 1-mercapto-2-propanol, 11-(1H-pyrrol-1-yl)undecane-1-thiol, 11-(ferrocenyl)undecanethiol, 11-azido-1-undecanethiol, 11-mercapto-1-undecanol, 11-mercaptoundecanoic acid, 11-mercaptoundecylhydroquinone, 12-mercaptododecanoic acid, 12-mercaptododecanoic acid NHS ester, 16-mercaptohexadecanoic acid, 3-amino-1-propanethiol hydrochloride, 3-chloro-1-propanethiol, 3-mercapto-1-propanol, 3-mercaptopropionic acid, 4-mercapto-1-butanol, 6-(ferrocenyl)hexanethiol, 6-amino-1-hexanethiol hydrochloride, 6-mercapto-1-hexanol, 6-mercaptohexanoic acid, 8-mercapto-1-octanol, 8-mercaptooctanoic acid, and 9-mercapto-1-nonanol; protected thiols comprising but not limited to [11-(methylcarbonylthio)undecyl]tri(ethylene glycol) methyl ether, [11-(methylcarbonylthio)undecyl]tri(ethylene glycol) acetic acid, [11-(methylcarbonylthio)undecyl]tetra(ethylene glycol), [11-(methylcarbonylthio)undecyl]hexa(ethylene glycol) methyl ether, hexa(ethylene glycol)mono-11-(acetylthio)undecyl ether, S-(10-undecenyl) thioacetate, S-(11-bromoundecyl) thioacetate, and S-(4-cyanobutyl)thioacetate; rings thiols comprising but not limited to cyclopentanethiol, cyclohexanethiol, thiophenol, m-carborane-1-thiol, p-terphenyl-4,4″-dithiol, biphenyl-4,4′-dithiol, 1,1′,4′,1″-terphenyl-4-thiol, 2-phenylethanethiol, 4-mercaptobenzoic acid, 1-naphthalenethiol, 4,4′-bis(mercaptomethyl)biphenyl, 9-mercaptofluorene, 1,4-benzenedimethanethiol, and 1-adamantanethiol; glutathione, mercaptopropionic acid (MPA), cysteine, cystamine, dihydrolipoic acid, and thiol-ending polyethylene glycol (PEG-SH).

In some examples, interacting molecules may be attached to metal particles using the electrostatic interactions between the interacting molecules and the metal particles, thus functionalizing and stabilizing metal particles bioconjugates. For example, gold particles are positively charged and bind by stable ionic interactions to negatively charged and nucleophilic moieties. In some examples, gold particles may interact with the phosphate ester backbone of nucleic acids, electron-dense regions of poly(amidoamine) dendrimers, or negatively-charged carboxylate groups.

In other examples, metal particles such as gold particles are efficiently stabilized by interacting molecules containing simultaneous acidic and basic groups (for example, proteins). Stabilization of metal particles, such as gold particles by interacting biological molecules may occur through passive adsorption of the interacting molecule onto the particle surface by electrostatic and hydrophobic interactions. A strong negative charge on a citrate-stabilized metal particle such as gold particle surface provides opportunity for Coulombic interaction with NH₂ groups of lysine residues of proteins adsorbed on the particle surface.

Advantageously, interacting molecules that are linked to metal particles via physical adsorption maintain their structure and function since the metal particles have a minimum effect on the interacting molecule. Accordingly, with native structure intact, the activity, selectivity, and specificity of the interacting molecule towards the nucleic acid binding molecule are largely unaffected.

Alternatively, in some examples, interacting molecules may also be attached to the shell of stabilizer molecules around the metal particles by bioconjugate chemistries, comprising but not limited to the covalent linkage of amino groups on the biological molecules with carboxyl groups at the free ends of stabilizer molecules by using 1-ethyl-3-(3-dimethylaminopropyl) carbodiimide HCl-mediated reaction, other “click chemistry”, biotin-streptavidin (SA) linking and azide-linking. Advantageously, such chemistries provide metal particles, such as gold nanoparticles molecule-specific binding sites.

As used herein, a protein in accordance with aspects of the invention may be referred to as a polynucleotide strand binding protein. The terms “protein”, “polynucleotide strand binding protein” and “nucleic acid binding protein” may be used interchangeably and may refer to any protein that binds nucleic acids. In some embodiments, the nucleic acid binding protein binds to double-stranded DNA in a sequence-specific manner, thus recognizing and binding to a specific sequence of nucleotides found within a double-stranded DNA molecule. The specific sequence to which the protein binds is referred to herein as a target recognition sequence or a target recognition site. The two terms may be used interchangeably. Many DNA-binding proteins and their particular consensus target recognition sequences are known. Examples of DNA-binding proteins include, without limitation, a transcription factors, including activators or suppressors of transcription, proteins involved in replication, or enzymes. The DNA-binding proteins bind dsDNA in a sequence-specific manner, and thus have a measurable binding affinity for target consensus sequences and may bind to sequences that have slightly altered sequences with a different (e.g., either greater or lesser) affinity.

The metal particles provided herein may be conjugated to a double-stranded (or partially double-stranded) DNA. The DNA may be conjugated to a particle by any method used to conjugate an organic molecule such as DNA to a metal. For example, free thiol groups react with metal, forming a bond between the metal surface and the sulphur atom. Thus, the dsDNA may be synthesized with an end group containing a free thiol on one end (e.g., the 5′ end) of one strand of dsDNA molecule, using standard laboratory techniques. The DNA molecule with a free thiol may be conjugated to the metal particle for use in any of the methods provided herein, thereby forming a thioether linkage between the metal particle and the DNA molecule as explained above.

In some embodiments, the metal particles conjugated to the interacting molecule are mixed (or combined or contacted) in a solution or reaction buffer and under conditions designed to provide appropriate conditions to allow for the cleaving molecule to cleave or catalyze the cleavage of the interacting molecule. Cleaving conditions for short DNA sequences with DNAse I are well known, including appropriate pH, temperature and ionic strength conditions.

In some embodiments, the solution or reaction buffer provides appropriate conditions (e.g., pH, ionic concentration and non-denaturing conditions) to provide for binding of the nucleic acid binding molecules to the target recognition sequence on the interacting molecule and for cleaving by the cleaving molecule. In some embodiments, there is provided a salt comprising a cation of Group 1 or 2 of the IUPAC periodic table of elements. In other embodiments, there is provided a salt comprising anions of Group 17 of the periodic table of elements.

In some embodiments, the reaction buffer contains an ionic species such as, for example, salt. Examples of salts include, without limitation, NaCl, CaCl₂, MgCl₂, KCl, MnCl₂, LiCl, Na₂CrO₄ or Na₂MoO₄ or mixtures thereof. The choice of salts may be based on their properties. For example, KCl is stronger than NaCl to aggregate particles. The charge of the salt may also influence the aggregation. For example, salts with higher charges such as MgCl₂ aggregate the metal particle more easily than KCl or NaCl. Thus, for example, the salt concentration may have a range of about 0.5 mM, 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, 50 mM, 60 mM, 80 mM, 100 mM, 150 mM, 200 mM, 300 mM, 400 mM, 500 mM, 750 mM, 1000 mM, 1500 mM, 2000 mM, 3000 mM, 4000 mM or 5000 mM or between about 0.5 to 5000 mM, about 5 to 500 mM, about 5 to 250 mM, about 5 to 150 mM, about 5 to 100 mM or about 10 to 100 mM.

In some embodiments, the reaction buffer may comprises phosphate buffered saline (PBS) or Tris-HCl. The ionic species may be selected to assist with the catalyzing conditions for the cleaving molecules. In addition, the ionic species may be selected for its ability to neutralize at least partially the charge present on the surface of the metal particles, resulting in aggregation of the particles upon cleavage of the interacting molecule.

As used herein, “ionic reaction buffer” and “solution” may be used interchangeably and may refer to reaction buffer that comprises an ionic species such as a salt at a concentration sufficient for metal particle aggregation.

In some embodiments, the reaction buffer comprises an effective amount of an ionic species sufficient to drive cleavage of the interacting molecules and concomitant aggregation of the particles. The ionic species may be, for example, any inorganic or organic salt. As will be appreciated, the precise identity and concentration of the ionic species selected for inclusion in the reaction buffer will depend on the nature of the particle used and the design of the DNA molecules and the complementary overhang regions. For example, different types of particles (e.g. gold versus silver) may have different lower limits for the concentration of ionic species in order to still aggregate upon annealing of the complementary overhang regions. Similarly, the same type of particle (e.g., gold) may have different lower limits for the concentration of ionic species, depending on the particular ionic species used and other buffer components (e.g., KCl versus Tris-HCl). In some embodiments, the ionic species used comprises KCl, NaCl, or CaCl₂ at a final concentration of 25 mM or greater in the reaction buffer. For example, the DNAse I buffer may comprise 10 mM Tris-HCl; 2.5 mM MgCl₂; 0.5 mM CaCl₂ and the pH may be 7.6 at 25° C. or the buffer may comprise 0.1 mM dithiothreitol; 6 mM MgCl₂; and 40 mM Tris-HCl at a pH of 7.5.

It is this combination of catalyzing and charge neutralization that results in aggregation of the metal particles in the reaction buffer in the absence of nucleic acid binding molecule. Metal particles conjugated with the interacting molecule will not aggregate in solution, even in the presence of the ionic species in the solution. Cleavage of the interacting molecule in the absence of sufficient concentration of ionic species may still result in aggregation of the metal particles, but any such aggregation will occur at a measurably slower rate than with concomitant charge neutralization.

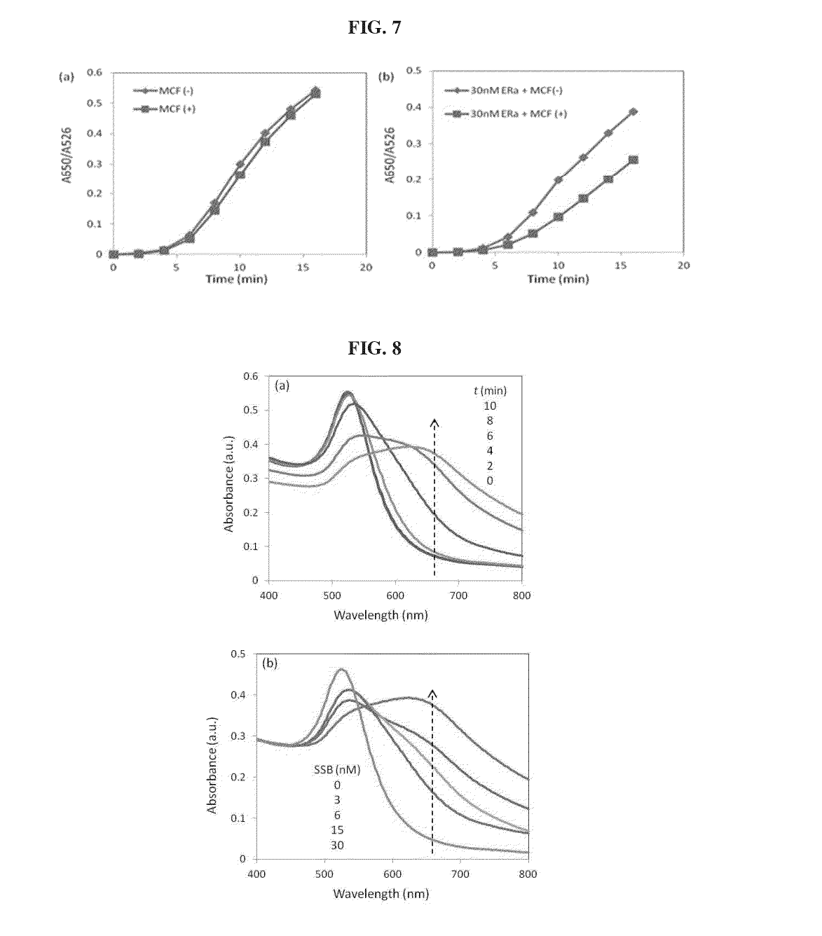

In addition, in some embodiments, the synergistic combination of cleavage and charge neutralization at the metal particle surface to induce aggregation of the metal particles, combined with the steric spacing affect of the polynucleotide strand binding molecule to reduce aggregation, allows for a rapid detection method that may not require long incubation periods. In some embodiments in case the concentration of polynucleotide strand binding molecules in the test sample is insufficient to avoid aggregation of metal particles after contacting with the cleaving molecule a known amount of polynucleotide strand binding molecules sufficient to bind to the metal particle and avoid aggregation is added to the mixture. This method is referred to herein as spike-in method as can be seen for example in example 3 below and FIG. 7 in the drawings.

The present inventors surprisingly found that in case of complex biological samples comprising numerous different polynucleotide strand binding molecules including the polynucleotide strand binding molecule of interest, the method as disclosed herein may not be sensitive enough to detect said polynucleotide strand binding molecule of interest. Thus in one example, there is provided the method as disclosed herein wherein the sample suspected to comprise the polynucleotide strand binding molecule of interest may be spiked with an amount of the isolated polynucleotide strand binding molecule of interest. The amount of polynucleotide strand binding molecule of interest to be added to the sample may be easily determined by the person skilled in the art.

For example, the amount of the polynucleotide strand binding molecule of interest may be added in the quantity sufficient to be detected using the metal particles-interacting molecules as described herein using the detection method described above. In one example, the amount of polynucleotide strand binding molecule of interest to be added to the sample may be about 10 nM, about 20 nM, about 30 nM, about 40 nM, about 50 nM or about 100 nM. The skilled person in the art would recognize that the amount to be added would depend on for example, the size of the sample, the composition of the sample, the concentration of the sample and of the metal particle-interacting molecules conjugates present in the solution.

It appears that spiking the sample comprising an undetectable amount of the polynucleotide strand binding molecule of interest with an isolated polynucleotide strand binding molecule may allow measurement of color differences in a sample comprising the native polynucleotide strand binding molecule of interest and a sample from the same origin where the native polynucleotide strand binding molecule of interest is absent. In one example, the sample may be a cell line extract such as a nuclear extract wherein the cell line does not express the nucleic acid binding molecule of interest. For example, the cell line may be a knock-out cell line for the nucleic acid binding molecule of interest.

A detection assay as described herein was demonstrated using estrogen receptor α as a model DNA-binding protein together with double-stranded DNA segments that form an estrogen response element conjugated to gold or silver NPs as the sensing platform. UV-vis spectroscopy, TEM, and dynamic light scattering (DLS) measurements were conducted to characterize the particle aggregation/dispersion mechanism.

Advantageously, the present method allows determination of the sequence specificity of DNA binding molecules. For example, the present method may be used to determine the binding specificity of small molecular DNA binding molecules. Many important drug candidates interact with DNA duplexes and triplexes to regulate gene expression by controlling transcription.

In the present invention, for example, the enzymatic control of the dispersion and aggregation of DNA coated AuNPs has been exploited. As will be seen below, the method as disclosed herein allows a fast colorimetric detection of nucleic acid (i.e. polynucleotide strand)-binding molecules by simple instrumentation (e.g., UV-Vis spectroscopy) and/or naked eyes observation owing to the interparticle distance-dependent LSPR properties of AuNPs. Advantageously, the hazardous radioactive-labeling and tedious assessment of cleavage pattern as required in the conventional footprinting assay is avoided.

In particular, the present method comprises the use of interacting molecules conjugated-metal particles in combination with a non-crosslinking mechanism (i.e., salt-induced aggregation of the particles) that renders the method specific and allows the detection of binding in minutes through solution color changes from red to blue. With the integration of enzymatic cleavage of DNA conjugated on the AuNPs surface, the method as disclosed in some of the examples below offers real-time monitoring of the enzymatic reaction and colorimetric detection of interaction between nucleic acid binding molecules such as proteins to interacting molecules such as DNA wherein the binding of said protein to said conjugated DNA is capable of inhibiting/hindering the enzymatic cleavage of the metal particles-conjugated DNA.

In the present method, the present inventors have developed a colorimetric sensing strategy for detecting both single- and double-stranded nucleic acid binding molecules. The method may comprise a combination of the conventional DNase I footprinting method with the highly-specific DNA modified-AuNPs sensing ability. Advantageously, a solution is prepared containing a solution that may be mixed with the sample comprising the nucleic acid binding molecule to be detected. Surprisingly, in case of interaction between the interacting molecule and the nucleic acid binding molecule, the solution color changes in about 5 minutes to about 60 minutes and gives visual and clear results. Advantageously, the method and the test format are in a homogenous solution, thus the method does not require complicated mixtures of different reagents such as different set of modified metal particles.

Beneficially, the method as disclosed herein the interacting molecules that are bound at the surface of the metal particles has no steric constraints that may affect the binding efficiency of the nucleic acid binding molecule that is to be detected. As will be seen below in some illustrative examples, DNA-conjugated AuNPs facilitate detection of nucleic acid binding molecules of interest that may be present in complex biological buffer or sample solution. Thus, the method as disclosed herein by using for example, DNA-conjugated AuNPs is more sensitive and selective than existing assay.

Interestingly, the method as disclosed herein may be used to detect for example, nucleic acid binding molecules that bind to molecules comprising but not limited to ssDNA and dsDNA as may be seen in Example 4 below. Furthermore, the nucleic acid binding molecule of interest may comprise but is not limited to drugs, metal ions, proteins, carbohydrates or lipids. As explained herein and in the following examples, the visual detection of color changes render the current method adapted for point-of-care diagnostics since no sophisticated equipment or detection facilities (e.g., radio protection lab) are required. Some other applications that may be envisaged by applying the current method comprise detection of cleavable molecule inhibitors, modulators or activators that may comprise but are not limited to changes in temperature, ionic concentration, pH of the buffer solution, small molecules, nucleic acid or protein capable of inhibiting the cleaving molecule as described herein and above.

The method as disclosed herein may also be used in identification of aptamers interacting molecules, small interfering RNA (siRNA) or microRNA (miRNA) inhibitors or other assays involving nucleic acid binding molecules and interacting molecules. Advantageously, the method as disclosed herein may be adaptable for example, to automation using illustratively 96 or 384-well microplate readers.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Example 1

### Aggregation Kinetics of dsDNA-Functionalized AuNPs in the Presence of DNase I

An application is described herein for detecting estrogen receptor (ER)-DNA interaction. ER is a DNA-binding protein that binds specifically to a dsDNA sequence known as estrogen-responsive elements (ERE), to regulate target gene transcription.

In this example, DNase I is used to cleave the DNA conjugated on the AuNPs surface to induce particle aggregation by the MgCl₂ and KCl content of the DNase I buffer. Double-stranded (ds) ERE sequences from the vitellogenin A2 gene (vit) containing the core binding sequence of 5′-GGTCAnnnTGACC-3′ (SEQ ID No. 1; n: spacer nucleotides) of ERα is conjugated on the AuNPs following the previously reported protocol. As shown in FIG. 2a, the as-conjugated dsvit-AuNPs is stable in DNase I buffer solution (20 mM MgCl₂ and 50 mM KCl) for at least 30 mM. When 5 unit/mL of DNase was added to the dsVit-AuNP in the same buffer solution, aggregation of AuNPs was observed, which corresponding the UV-vis spectrum shift to the longer wavelength (FIG. 2b). The time-dependent aggregation of AuNPs is induced by the MgCl₂ and KCl content of the DNase I buffer due to the DNase cleavage of dsVit on the AuNPs surface and lost the electrosteric protection of DNA to AuNPs from charge screening by salt ions. All the DNase experiments are carried out at 37° C. to achieve optimal enzymatic activity.

In order to demonstrate the ability of DNase I to reduce dsVit-AuNPs stability, different concentrations of DNase I were tested. FIG. 3 show that increasing concentration of DNAse I in the dsvit-AuNPs solution accelerates the catalytic reaction (that is the cleavage of the dsVit from the AuNPs). Accordingly the kinetic of AuNPs aggregation was enhanced when the amount of DNAse I was increased. The kinetic of AuNPs was observed for one hour. The amount of DNase I can thus be manipulated (i.e., 20 unit/mL of DNase I) to enable a rapid detection of DNase I activity in less than 5 minutes by color changes from red (dispersed AuNPs) to blue (aggregated AuNPs).

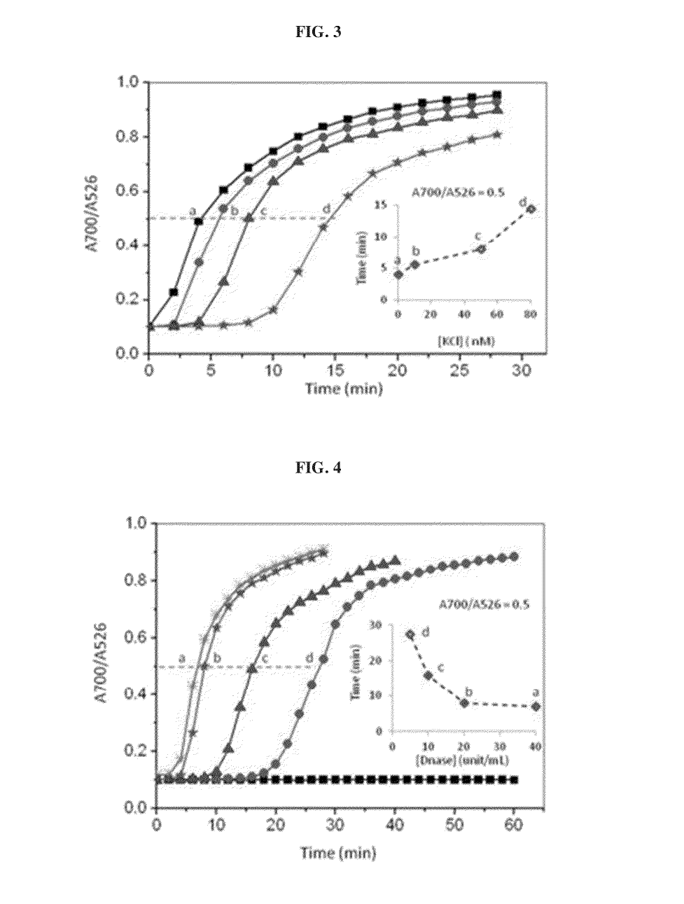

To further prove that the aggregation of AuNPs is related to the enzymatic activity of DNase I and the ability of the enzyme to cleave the protective DNA conjugated from the AuNPs surface, the present inventors have carried out a salt test using KCl to tune the enzymatic activity of DNase I. It is well known that DNase I activity is sensitive to the salt concentration in the buffer solution. A salt concentration of more than 100 mM reduces DNase I activity. FIG. 4 shows the aggregation kinetics of dsVit-AuNPs in the presence of 20 unit/mL of DNase I in a buffer solution comprising 20 mM MgCl₂ and KCl at concentrations ranging from 0 to 80 mM (0, 10, 50 and 80 mM). By comparing the time necessary for the samples to reach an aggregation extent of 0.5 (that is the ratio of A₇₀₀/A₅₂₆; inset of FIG. 4), it is shown that a lower concentration of KCl in the buffer solution containing dsvit-AuNPs results in a faster aggregation kinetic. For example, when dsvit-AuNPs were incubated in the presence of DNAse I in a buffer solution containing 80 mM KCl, it took 15 mM to reach an aggregation extent of 0.5. Conversely when the dsvit-AuNPs were incubated in a solution containing the same amount of DNAse I but 10 mM KCl, the same aggregation extent of 0.5 was reached in 5 minutes. This shows that the activity of DNase I is reduced in the presence of a higher KCl concentration and that the lower DNAse I activity affects the catalytic cleavage rate of the DNA conjugated to the AuNPs surface, leading to a slower (or less extensive) particle aggregation.

### Example 2

### Detection of ERα Using dsDNA-Modified AuNPs and DNase

In the example 1 above, it has been shown that the aggregation of AuNPs resulting from the enzymatic cleavage of the DNA conjugated to the particle surface can be detected immediately by naked eyes observation of color changes and/or quantified by the absorption spectra measurement. Using the ERα and their DNA response elements as a model system (FIG. 5a), the inventors demonstrated that the presence of ERα and its binding to the specific DNA on AuNPs can be detected based on the retarded/absent DNA cleavage, which in turn keeps the nanoparticles dispersed (and thus red) under high salt conditions, while the sample without ERα experienced extensive aggregation (purple) in the same salt and DNase I concentration. To examine the effects of the ERα protection on AuNPs, concentration of ERα was varied with a fixed amount of DNase I (20 units/mL). FIG. 5b shows that decreasing concentration of ERα in the sample results in an increase, the aggregation extent of AuNPs is increased. The lower limit of detection using the present method is about 10 nM of ERα. This limit is lower (that is the present nanoparticle assay is more sensitive) than a previously reported colorimetric assay for ERα detection that uses unmodified AuNPs as the sensing elements.

To check the specificity of this assay, the inventors used bovine serum albumin (BSA), a protein not capable of binding DNA, as a control. BSA was mixed together with ERα at different concentration ratio to form a protein mixture having a total concentration of 40 nM. The color shift of the DNA-conjugated AuNPs was recorded at time interval of 1, 5 and 8 minutes for each sample as indicated in FIG. 6a. It was observed that the AuNPs sample comprising only BSA (40 nM; and 0 nM ERα) changes color (from red) to purple after a 5 minutes DNase I treatment. This result shows that BSA does not bind inhibit the cleavage of the DNA conjugated to the AuNPs surface; in other words, BSA cannot protect the DNA strand from DNase I digestion, leading to extensive particle aggregation in the buffer solution containing 20 mM MgCl₂ and 50 mM KCl. That is, it does not appear that BSA binds to the DNA conjugated to the AuNPs. On the other hand, samples comprising different amount of ERα (10 nM, 20 nM, 30 nM and 40 nM) in the protein mixture exhibited stabilization effects similar to those achieved by ERα alone as shown in FIG. 6b. These results demonstrate that the instant nanoparticle assay is highly specific in its detection ability. The present method is more convenient and sensitive than previously reported colorimetric assay for detection of DNA-binding proteins.

### Example 3

### Detection of ERα in Nuclear Extracts from MCF-7 Human Breast Cancer Cell

Nuclear extracts contain thousands of different transcription factors. It thus renders detection and identification of a DNA binding proteins of interest, particularly challenging in terms of selectivity and specificity. Sure enough, the method should allow specific binding of one particular proteins among a mixture of thousand others different DNA binding proteins in a physiological setting (i.e. the nucleus of isolated cells)

The inventors used the DNase-assisted AuNPs assay to detect the binding of native (i.e. as opposed to recombinant or ERα from human MCF-7 breast cancer cells extracts to its DNA response element. It was found that the usage of dsvit-AuNPs alone failed to detect the ERα-DNA binding (FIG. 7a) upon addition of DNase, due to insufficient amount of native ERα expression in the biological samples. However, the MCF (ER+) sample is differentiable from the MCF (ER−) sample when 30 nM of recombinant ERα was spiked into the nuclear extract samples (FIG. 7b).

### Example 4

### Detection of Single-Stranded DNA Binding (SSB) Protein Using ssDNA-Modified AuNPs and DNase

To demonstrate the versatility of current assay, the present inventors extended the detection to the single-stranded DNA binding protein (SSB). SSB is a protein that binds to single-stranded regions of DNA to prevent premature annealing to protect the ssDNA from being digested by nucleases, and to remove secondary structure from the DNA to allow other enzymes to function effectively upon it. In this example, the protection effect of SSB was tested to protect ss-vit from DNase I digestion. As shown in FIG. 8a, the antisense-conjugated ssvit-AuNPs aggregate upon addition of 20 unit/mL of DNase I in the 20 mM as-conjugated ssvit-AuNPs undergo extensive aggregation upon addition of 20 unit/L of DNAse in the 20 mM MgCl₂ and 50 mM KCl buffer solution. The presence of SSB retarded the aggregation extent of ssvit-AuNPs in a concentration dependent manner (FIG. 8b), which assay can be used to detect the SSB as low as 3 nM.

1. A colorimetric method of detecting a polynucleotide strand binding molecule, wherein the method comprises:
i. contacting a sample suspected to comprise a polynucleotide strand binding molecule with a solution comprising one type of metal particles to obtain a sample-particle mixture, wherein the metal particle comprises at its surface a single type of interacting molecules capable of specifically binding to a specific polynucleotide strand binding molecule and capable of protecting the metal particle from aggregation;
ii. incubating the mixture for a time sufficient to allow binding of the polynucleotide strand binding molecule to the interacting molecule;
iii. contacting the mixture with a cleaving molecule capable of cleaving the interacting molecule;
iv. incubating the mixture of iii. for a time sufficient to allow cleavage of the interacting molecule; and
v. measuring color differences wherein a color difference compared to the initial color of the mixture due to metal particle aggregation indicates that the polynucleotide strand binding molecule is not present in the sample;
wherein a salt is either mixed into the mixture together with the sample or is already present in the solution comprising the metal particle or is added together with the cleaving molecule.

2. The method of claim 1, wherein the interacting molecule is a polynucleotide molecule.

3. The method of claim 2, wherein the polynucleotide molecule is a double stranded or single stranded polynucleotide molecule.

4. The method of claim 1, wherein the interacting molecule is selected from the group consisting of a deoxyribonucleic acid (DNA), a ribonucleic acid (RNA), a small interfering RNA, a micro RNA and an aptamer.

5. The method of claim 1, wherein the metal particle is made of a noble metal, core-shell or alloys of noble metals.

6. The method of claim 5, wherein the noble metal is any one of gold, silver, palladium, ruthenium, rhodium, osmium, iridium or platinum.

7. The method of claim 6, wherein the noble metal is gold or silver.

8. The method of claim 1, wherein the metal particle is a nanoparticle or a microparticle having a size comprised between at least about 3 nm to at least about 1000 nm.

9. The method of claim 1, wherein protection of the metal particle in step i further comprises functionalizing the metal particle with a molecule selected from the group consisting of a nucleic acid, a peptide, a protein, a polymer including polyethylene glycol (PEG) and a charged molecule comprising sodium citrate, a single nucleotide or ATP.

10. The method of claim 1, wherein the salt is selected from salts comprising a cation of Group 1 or 2 of the IUPAC periodic system.

11. The method of claim 1, wherein the salt is selected from salts comprising anions of Group 17 of the periodic system.

12. The method of claim 9, wherein the salt is selected from the group consisting of NaCl, CaCl2, MgCl2, KCl, or MnCl2 or mixtures thereof.

13. The method of claim 1, wherein measuring of color differences comprises one or more of microscopy techniques, dynamic light scattering techniques, visual observation of color, UV/Vis absorption techniques, or surface plasmon resonance techniques.

14. The method of claim 12, wherein a surface plasmon resonance technique is a localized surface plasmon resonance technique (LSPR).

15. The method of claim 1, wherein the interacting molecule is a double stranded or single stranded nucleotide molecule and the nucleotide strand binding molecule is a nucleotide binding protein.

16. The method of claim 1, wherein the cleaving molecule is an enzyme or any molecule capable of cleaving the interacting molecule

17. The method of claim 15, wherein the enzyme is a nuclease, wherein the nuclease comprises an endonuclease or an exonuclease.

18. The method of claim 17, wherein the nuclease is selected from the group consisting of a Deoxyribonuclease (DNase), a Ribonuclease (RNase), an endo VI, an exo III, a T7 endonuclease and a S1-nuclease.

19. The method of claim 18, wherein the DNase is DNase I.

20. The method of claim 1, wherein the interacting molecule is bound to the surface of the metal particle via a linker, ionic attachment, chemisorption or physical adsorption.

21. The method of claim 20, wherein the linker is selected from the group comprising C₁-C₂₀ alkyl thiol selected from the group comprising 1-propanethiol, 1-butanethiol, 1-pentanethiol, 1-hexanethiol, 1-heptanethiol, 1-octanethiol, 1-nonanethiol, 1-decanethiol, 1-undecanethiol, 1-dodecanethiol, 1-tetradecanethiol, 1-pentadecanethiol, 1-hexadecanethiol, 1-octadecanethiol, 2-ethylhexanethiol, 2-methyl-1-propanethiol, 3-methyl-1-butanethiol, butyl 3-mercaptopropionate, tert-dodecylmercaptan, and tert-nonylmercaptan; dithiols comprising but not limited to 1,2-ethandithiol, 1,3-propanedithiol, 1,4-butanedithiol, 2,3-butanedithiol, 1,5-pentanedithiol, 1,6-hexanedithiol, 1,8-octanedithiol, 1,9-nonanedithiol, tetra(ethylene glycol) dithiol, hexa(ethylene glycol) dithiol, 2,2′-(ethylenedioxy)diethanethiol, and 5,5′-bis(mercaptomethyl)-2,2′-bipyridine; functionalized thiols selected from the group comprising (11-Mercaptoundecyl)-N,N,N-trimethylammonium bromide, 1-Mercapto-2-propanol, 11-(1H-pyrrol-1-yl)undecane-1-thiol, 11-(Ferrocenyl)undecanethiol, 11-Azido-1-undecanethiol, 11-Mercapto-1-undecanol, 11-Mercaptoundecanoic acid, 11-Mercaptoundecylhydroquinone, 12-Mercaptododecanoic acid, 12-Mercaptododecanoic acid NHS ester, 16-Mercaptohexadecanoic acid, 3-Amino-1-propanethiol hydrochloride, 3-Chloro-1-propanethiol, 3-Mercapto-1-propanol, 3-Mercaptopropionic acid, 4-Mercapto-1-butanol, 6-(Ferrocenyl)hexanethiol, 6-Amino-1-hexanethiol hydrochloride, 6-Mercapto-1-hexanol, 6-Mercaptohexanoic acid, 8-Mercapto-1-octanol, 8-Mercaptooctanoic acid, and 9-Mercapto-1-nonanol; protected thiols selected from the group comprising [11-(Methylcarbonylthio)undecyl]tri(ethylene glycol) methyl ether, [11-(Methylcarbonylthio)undecyl]tri(ethylene glycol) acetic acid, [11-(Methylcarbonylthio)undecyl]tetra(ethylene glycol), [11-(Methylcarbonylthio)undecyl]hexa(ethylene glycol) methyl ether, Hexa(ethylene glycol)mono-1′-(acetylthio)undecyl ether, S-(10-Undecenyl) thioacetate, S-(11-Bromoundecyl) thioacetate, and S-(4-Cyanobutyl)thioacetate; rings thiols selected from the group comprising cyclopentanethiol, cyclohexanethiol, thiophenol, m-carborane-1-thiol, p-terphenyl-4,4″-dithiol, biphenyl-4,4′-dithiol, 1,1′,4′,1″-terphenyl-4-thiol, 2-phenylethanethiol, 4-mercaptobenzoic acid, 1-naphthalenethiol, 4,4′-bis(mercaptomethyl)biphenyl, 9-mercaptofluorene, 1,4-benzenedimethanethiol, and 1-adamantanethiol; glutathione, mercaptopropionic acid (MPA), cysteine, cystamine, dihydrolipoic acid, and thiol-ending polyethylene glycol (PEG-SH).

22. The method of claim 20, wherein the interacting molecule is bound to the linker by a method comprising covalent binding of an amino group of the interacting molecule to a carboxy group of the linker by 1-ethyl-3-(3-dimethylaminopropyl) carbodiimide HCl-mediated reaction (EDC), biotin-avidin linkage, or click chemistries including azide-linkage.

23. The method of claim 20, wherein chemisorption is via a functional group which bind the metal particle, wherein the functional group selected from the group consisting of thiols and peptide sequences comprising cysteine and thiolated amino acids.

24. The method of claim 20, wherein physical adsorption comprises binding of peptide sequences comprising simultaneous acidic and basic amino acids groups.

25. The method of claim 1, wherein in case the concentration of nucleotide strand binding molecules in the test sample is insufficient to avoid aggregation of metal particles after contacting with the cleaving molecule a known amount of nucleotide strand binding molecules sufficient to bind to the metal particle and avoid aggregation is added to the mixture in step iv.

26. The method of claim 1, wherein the detection is a qualitative colorimetric method of detecting a nucleotide strand binding molecule or a qualitative and quantitative colorimetric method of detecting a nucleotide strand binding molecule.

27. The method of claim 1, wherein metal particle aggregation is obtained via salt aggregation and/or cleavage of the interacting molecule.


SEQUENCE LISTING (2 sequences)

SEQ ID NO: 1 - DNA, 13 bp, Homo sapiens
  spacer nucleotides (6)..(8): n can be any nucleotide and serve as a spacer in the core binding sequence of the estrogen-responsive element.
ggtcannntgacc

SEQ ID NO: 2 - DNA, 13 bp, Homo sapiens
ggtcacagtgacc